(19) United States
(12) Patent Application Publication
(10) Pub. No.: US 2008/0153775 A1
(43) Pub. Date: Jun. 26, 2008

(54) WEIGHT REDUCTION PROGRAM AND METHOD

(76) Inventor: Floyd S. Dillard, Eustis, FL (US)

Correspondence Address: EDWARD M. LIVINGSTON, PA, 963 TRAIL TERRACE DRIVE, NAPLES, FL, 34103, US

(21) Appl. No.: 11/845,131

(22) Filed: Aug. 27, 2007

Related U.S. Application Data
(60) Provisional application No. 60/871,370, filed on Dec. 21, 2006.

(51) Int. Cl. A61K 31/714; A61K 31/135; A61P 3/00; A61K 31/19

(52) U.S. Cl. 514/52; 514/557; 514/646

(57) ABSTRACT

A new weight reduction program and method 1 having three phases, an acute weight loss phase 2, a transitional phase 3 and a long-term maintenance phase 4. Appetite suppressants 12 (such as phenylkylamine sympathomimetic amine 13), calcium pyruvate 17, a multiple vitamin-mineral supplement 16, amino acids and cyanocobalomine 10 are taken in conjunction with the new diet program to achieve optimal weight loss and health benefits.

### CROSS REFERENCE TO RELATED APPLICATION

This application claims the benefit of U.S. Provisional Application No. 60/871,370, filed Dec. 21, 2006.

### BACKGROUND OF THE INVENTION

The present invention relates to a new diet program and method that creates a healthier body by providing a three-phase program.

About two-thirds of the adult population of the United States is overweight. Of this amount, almost one-third is clinically obese, or is at least twenty percent (20%) or greater over his/her ideal weight. In the 1990's, rates of obesity more than doubled and increasing rising by over five percent (5%) each year. Excess body weight is implicated as a risk factor for many different disorders, including, but not limited to, heart disease, diabetes, cancer, strokes, heart attacks, heart failure, prostate enlargement (BPH), female infertility, uterine fibroids, gallstones, gout, osteoarthritis, sleep apnea, dementia and other disorders of pregnancy, including, but not limited to, birth defects, gestational diabetes, preeclampsia and gestational hypertension.

In addition, it is not only the amount of body fat that has adverse effects on one's health, but it is also the location of the excess body fat. For instance, increased abdominal fat, which leads to an “apple-shape”, may be especially hazardous to long-term health as the deposit of fat around the abdomen leads to higher risks of the aforementioned disorders when compared to increased amounts of body fat around the hips and thighs, which leads to a “pear-shape.”

According to most short-term studies, the effect of exercise alone, that is, without any type of dietary restriction, on weight loss is small, partly because muscle mass, which is denser than fatty tissue, often increases, thereby resulting in a greater weight. In addition, it is often found that individuals who exercise experience an increase in appetite, thereby actually consuming more calories as compared to his/her sedentary state. Although the long-term effect of regular exercise on weight loss is great and aids in maintaining an ideal weight, it is individuals who combine a healthy diet with an exercise regime that experience the greatest health benefits, which include a long, active life with minimal serious diseases.

Although the importance of a healthy, well-balanced diet and exercise are well-known, many individuals still find it difficult to lose weight due to various factors, including time restraints and periodic conflicting studies concluding what types of foods are healthy and in what amounts. In addition, many individuals undertake drastic changes in his/her diet without consulting a physician, thus potentially causing oneself to limit or even eliminate the vitamins and minerals which a body needs to function properly, ironically causing even greater health problems to the individual than being overweight or obese.

Thus, a need for a safe, new diet program that incorporates a healthy diet and exercise program is needed.

The relevant prior art includes the following patents:

| Patent No. | | |
|---|---|---|
| (U.S. unless stated otherwise) | Inventor | Issue Date |
| 2006/0051435 | Udell et al. | Mar. 09, 2006 |
| 7,078,537 | Choi et al. | Jul. 18, 2006 |
| 2006/0025476 | Antosh | Feb. 02, 2006 |
| 6,277,842 | Carthron | Aug. 21, 2001 |
| 2006/0062764 | McCleary | Mar. 23, 2006 |
| 2006/0183803 | Hevia et al. | Aug. 17, 2006 |
| 6,210,702 | Samman | Apr. 03, 2001 |
| 7,053,075 | DeLuca et al. | May 30, 2006 |

### SUMMARY OF THE INVENTION

The primary object of the present invention is to provide a new diet program that is safe and effective long term.

Another object of the present invention is to provide a new diet program that causes an individual to lose weight.

A further object of the present invention is to provide a new diet program that promotes healthy eating and a healthy lifestyle.

An even further object of the present invention is to provide a new diet program that promotes exercise in conjunction with weight loss to tone muscle, providing long term sustained weight loss.

Another object of the present invention is to provide a new diet program that is monitored by medically-trained personnel.

An even further object of the present invention is to provide a new diet program that provides various nutritional supplements so as to maintain optimal health.

The present invention fulfills the above and other objects by providing a new diet program having a three-phase or three-step program wherein an acute weight loss phase, a short-term maintenance or transitional phase and a long-term maintenance phase are utilized. Appetite suppressants (such as phenylkylamine sympathomimetic amine), calcium pyruvate, a multiple vitamin-mineral supplement, amino acids and cyanocobalomine are taken in conjunction with the new diet program to achieve optimal weight loss and health benefits.

The above and other objects, features and advantages of the present invention should become even more readily apparent to those skilled in the art upon a reading of the following detailed description.

### BRIEF DESCRIPTION OF THE DRAWINGS

In the following detailed description, reference will be made to the attached drawings in which:

FIG. 1 is a flow diagram representing the phases of the present invention;

FIG. 2 is a flow diagram representing an acute weight loss phase of the present invention;

FIG. 3 is a flow diagram representing a transitional phase of the present invention; and

FIG. 4 is a flow diagram representing a long-term maintenance phase of the present invention.

### DESCRIPTION OF THE PREFERRED EMBODIMENTS

For purposes of describing the preferred embodiment, the terminology used in reference to the numbered components in the drawings is as follows:
1. weight reduction method, generally
2. acute weight loss phase
3. transitional phase
4. long-term maintenance phase
5. blood work
6. weight monitoring
7. blood pressure monitoring
8. exercise
9. nutritional consultation
10. cyanocobalamine
11. electrocardiogram
12. appetite suppressant
13. phenylalkylamine sympathomimetic amine
14. anoretic
15. anorexigenic
16. vitamin-mineral supplement
17. calcium pyruvate

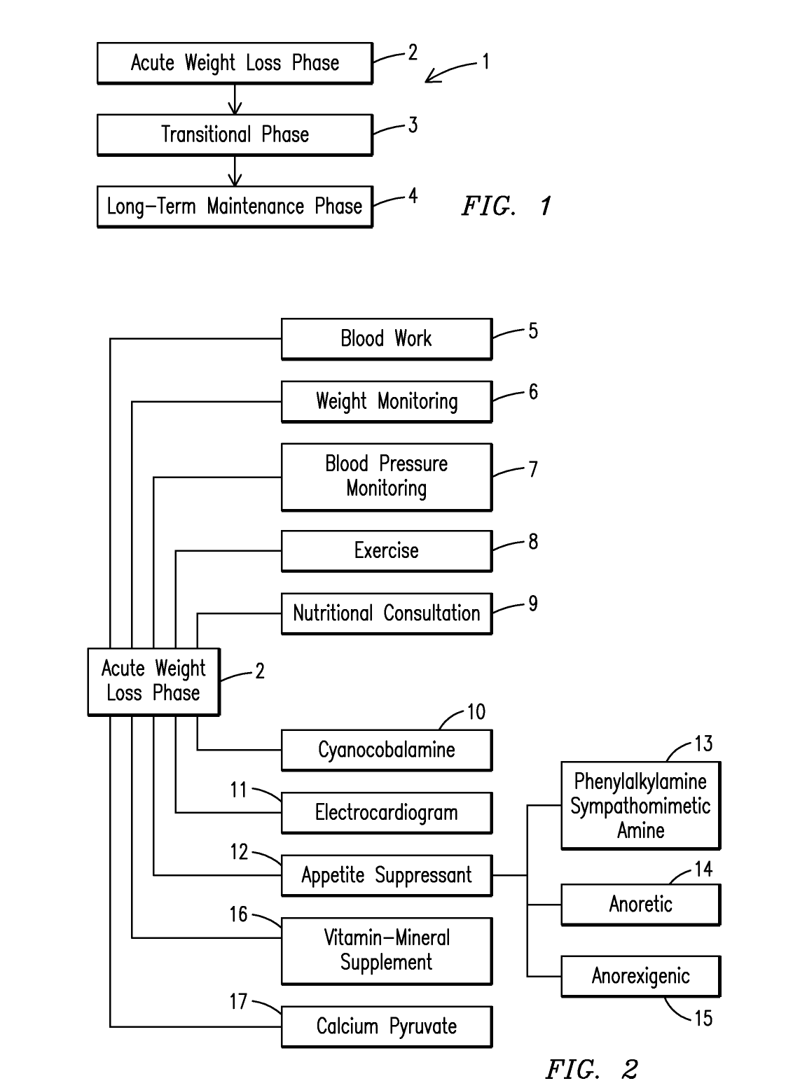

With reference to FIG. 1, a flow diagram representing the phases of the present invention is shown. The weight reduction method, generally 1 consists of an acute weight loss phase 2, a transitional phase 3 and a long-term maintenance phase 4.

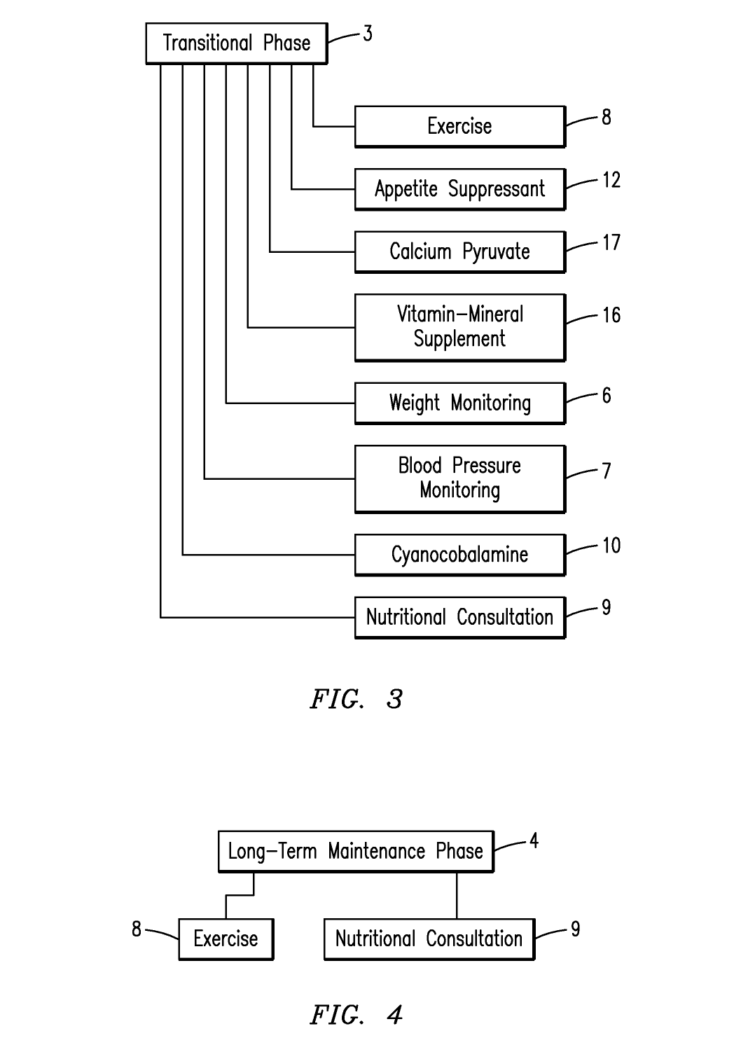

With reference to FIGS. 2-4, flow diagrams of the acute weight loss phase 2, a transitional phase 3 and long-term maintenance phase 4 are shown.

The present invention relates to a new diet program wherein a healthy diet, including supplements, and an exercise regime is followed to achieve maximum and long-lasting weight loss wherein a person's blood insulin level is lowered. The program is divided into three distinct phases: phase one for acute weight loss, phase two for short-term maintenance and phase three for long-term maintenance. The entire program is preferably monitored by medically-trained personnel so as to ensure the user's peak health by monitoring the blood pressure, weight and diet of the user, as well as assessing any health risks taken by the user. In addition, the personnel educates the user as to which food products he/she should eat and the food products which naturally lower serum glucose, which may decrease the risks of birth defects, some cancers, heart disease and dementia.

With respect to phase one of the new diet program 1 of the present invention, also known as the acute weight loss phase 2, acute weight loss is the goal. Initial blood work 5, weight and blood pressure monitoring 6 and 7, respectively, exercise 8 and nutritional consultation 9 and a predetermined amount of cyanocobalomine (B12) 10 are administered by the medical personnel, the latter of which is preferably via injection. An electrocardiogram 11, or EKG, may also be administered by medical personnel at this time as deemed necessary.

During the first five days of the new diet program, the user is placed on a restricted diet and must eat three times a day. The goal during the first five days of the new diet program is to induce the body into ketosis, a process wherein the body burns fatty tissue. Ketosis is achieved by consuming a diet that consists of protein only. The new diet program permits the following items as sources of protein: lean meats, including steak, roast beef, hamburger, pork chops, cooked ham, ribs, lamb, veal and deli meats; poultry, including chicken, turkey, duck, ground turkey, ground chicken and deli meats; fish, including salmon, tuna, swordfish, flounder, haddock, halibut, cod, orange roughy, sole, sea bass, turbot, smoked fish and lox; and seafood, including shrimp, scallops, lobster, crab and oysters; eggs; and cottage cheese. All fat and skin should be removed from the meats prior to cooking and eating. The user is only permitted to eat eight hundred calories per day (unless otherwise prescribed by medical personnel) during the first five days unless otherwise prescribed wherein the calories should preferably be divided into three meals per day. Absolutely no fruit, vegetables, juice, coffee creamer, candy, breath mints, alcohol, breads or sugared gums may be consumed during the first five days. However, if the user should feel weak, fatigued or short of breath, he or she should contact medical personnel and add a piece of fruit, such as ½ an apple, between meals to insure that the user has not advanced too far into ketosis.

Salt, pepper, herbs, spices, dry barbeque spice, cocktail sauce, lemon juice, lime juice, ketchup, mustard, Miracle Whip® Free, fat free mayonnaise, butter and cinnamon may be used. In addition, Equal® or Sweet'n Low® packets, may be used as well.

After the first five days, the user should be in moderate ketosis wherein he or she should continue to eat three meals a day with snacks wherein carbohydrates, fats and sugar are limited and protein and vegetable intake is high. During this time, the user is still not permitted to eat beets, breads, carrots, corn, pasta, potatoes, rice or tomatoes but is permitted to use any salad dressing having less than two grams of sugar and less that two grams of carbohydrates per serving.

In addition, the user is permitted to eat two servings of fruits, such as apples, cherries, grapes, grapefruit, oranges and strawberries, and two servings of vegetables, such as asparagus, broccoli, brussel sprouts, cabbage, cauliflower, celery, collard greens, cucumbers, green beans, kale, lettuce, mushrooms, onions, spinach, sprouts, string beans, sauerkraut, dill pickles and baked sweet potatoes (the latter of which should be only two per week). The user should also drink one gallon of water per day. The user should eliminate consumption of alcohol or at least severely limit the use of alcohol.

With regards to snacking during the first phase of the program, the user may eat six almonds or walnuts as they are good sources of protein. In addition, pine nuts, berries, sugar-free and fat-free yogurt, skim milk and fat-free milk may also be utilized for snacking purposes.

A predetermined amount of an appetite suppressant 12, preferably phenylalkylamine sympathomimetic amine 13, is taken when the user is participating in the first phase of the program. The pharmacological actions of the appetite suppressant include central nervous system stimulation and elevation of blood pressure. Drugs of this class are commonly referred to as anorectics 14 or anorexigenics 15. The action of such drugs in treating obesity is primarily one of appetite suppressions, however, other central nervous system actions or metabolic effects may be involved.

In addition, a predetermined amount of at least one multiple vitamin-mineral supplement 16 is taken while the user is participating in the first phase of the new diet program so as to ensure the user is receiving the proper recommended daily dosage of vitamins and minerals.

Furthermore, a predetermined amount of calcium pyruvate 17 is also taken, preferably via ingestion, by the user when participating in the first phase of the program. Pyruvate is a compound that occurs naturally in the body. The inclusion of calcium pyruvate 17 in one's diet has many positive benefits, including improving one's sports ability, increasing the content of cell energy, reducing or even preventing more fat or weight gain, controlling partial lack of blood when the heart has been injured, strengthening the function of the heart muscle, decreasing cholesterol and the amount of damage to a heart caused by sudden disease, lowering blood sugar, alleviating eye sickness of a diabetic individual and preventing free radicals from growing and spreading.

The user of the program should visit with his/her medical personnel on a weekly basis so as to receive weekly injections of cyanocobalomine (B12) 10, receive one week supply of appetite suppressant 12, as well as having his/her weight and blood pressure monitored 6 and 7, respectively and receive nutritional consultation 9 and instruction. The medical personnel, such as a physician, physician's assistant or a nurse practitioner, monitors clients who are on diabetic and/or antihypertension medications to adjust dosing as the weight loss and healthy diet decrease the need for these medications dramatically and quickly.

Finally, if the user of the program finds himself/herself sluggish, medical personnel may administer cyanocobalomine 10 (B12) via an injection, as well as a B6 injection, to boost energy and prolong the effectiveness of B12 for clients who have reached a plateau or who are going on vacation in an effort to maintain the metabolism boost and continue the fat burning process. The inclusion of cyanocobalomine 10 has been considered to have a possible medical link to decreasing the risk of dementia and arteriosclerosis.

The user remains on the first phase of the program until his/her ideal weight is attained, at which point he/she transitions into phase two of the program.

Phase two of the program is referred to as the short-term maintenance phase or the transitional phase 3 of the program. This transitional phase 3 is where the user is instructed as to how to maintain the nutritional program of the present invention via nutritional consultation 9 and wherein greater emphasis is placed on the user's activity level, preferably requiring a predetermined amount of exercise 8 to be performed. During this phase of the program, the user should maintain the amount of weight loss achieved in the first phase for a predetermined amount of time, preferably one month. The amount of appetite suppressant 12 is decreased by half for the first two weeks of the last month of the transitional phase wherein a user takes one pill three times a day instead of two pills three times a day; however, the user is encourage to continue taking the same amount of calcium pyruvate 17 and vitamin-mineral supplement 16 as in the first phase of the program. Weight and blood pressure monitoring 6 and 7, respectively, is conducted every two weeks during the transitional phase 3. After the first two weeks of the month-long transitional phase 3, the user receives a final injection of cyanocobalomine 10 (B12) and a final dose of appetite suppressant 12. If the user gains weight within a four week time span, then he/she remains on the transitional phase 3 but increases the amount of physical activity/exercises 8.

The recommended exercises 8 during the transitional phase 3 include aerobic exercise 8, such as walking, jogging, bicycling, swimming and cardiovascular exercise 8 on an elliptical trainer, resistance exercises, such as weight training, and flexibility exercises 8, such as yoga, pilates and stretching.

Aerobic exercise 8 is of great importance as burns calories and fat, increases blood flow to the heart and throughout the body and strengthens the heart. Aerobic exercises 8 should be performed at least thirty minutes three times a week. Each session should be strenuous, but not overly excessive so as to cause injury to the user.

The inclusion of resistance exercise 8 is of great importance as it promotes healthy muscle and bone growth. The weights should be heavy enough to challenge the muscle group being worked in order to promote growth of the muscle tissue. All of the muscle groups should be worked at different sessions, i.e., chest, arms and shoulders in one session, legs, calves and back in another session. Abdominal muscles can be exercised daily.

Flexibility exercises 8 are useful in maintaining the flexibility of muscles and joints in order to prevent injury and to maintain function of the muscles and joints throughout the user's lifetime.

If, after four weeks, the user has gained no weight, then the user moves on to phase three, the long-term maintenance phase 4.

The third and final phase, the long-term maintenance phase 4, is of great importance since the majority of individuals require continuous monitoring so as to maintain his/her weight loss. During this phase of the program, continued nutritional consultation 9 and exercise 8 is stressed. This phase of the program is implemented only if necessary for the user's continued weight loss success.

The long-term maintenance phase 4 allows a person to maintain his/her goal weight that was achieved by following the first two phases by consuming an amount of calories that is no more than ten times the user's goal weight per day, i.e., a goal weight of 150 pounds multiplied by ten equals 1,500 calories per day, the amount of calories required to maintain the goal weight. This amount of caloric intake is calculated before activity and exercise.

During the long-term maintenance phase 4, alcohol and desserts may be consumed. If consumed, however, the amount of caloric intake in the morning and in the afternoon should be decreased and the amount of exercise 8 should be increased. Meals should include fifty percent carbohydrates, forty to forty-five percent protein and five to ten percent fats wherein trans-fats are avoided. If sugar is to be consumed, light brown or raw sugar should be used, as opposed to white processed or synthetic sugars. In addition, the following foods should be limited: bleached white flower products, white starches, white sugar, etc. and any processed foods.

Exercise 8 is encouraged during all phases of the program. In addition, vital signs and weight monitoring 6 is conducted by medical personnel throughout the entire program.

Although only a few embodiments of the present invention have been described in detail hereinabove, all improvements and modifications to this invention within the scope or equivalents of the claims are included as part of this invention.

Having thus described my invention, I claim:

1. A weight reduction program for losing a predetermined amount of weight and maintaining a loss of said predetermined amount of weight comprising:
an acute weight loss phase wherein a user enters ketosis;
a predetermined amount of appetite suppressant is taken during said acute weight loss phase;
a predetermined amount of at least one multiple vitamin-mineral supplement is taken during said acute weight loss phase;
a transitional phase wherein said predetermined loss of said predetermined amount of weight is maintained for a predetermined amount of time;
a predetermined amount of exercise is performed during said transitional phase; and
a long-term maintenance phase wherein a predetermined amount of calories equaling no more than ten times a user's goal weight per day is consumed.

2. The weight reduction program of claim 1 wherein:
said at least one appetite suppressant is phenylalkylamine sympathomimetic amine.

3. The weight reduction program of claim 1 wherein:
said at least one appetite suppressant is an anorectic.

4. The weight reduction program of claim 1 wherein:
said at least one appetite suppressant is an anorexigenic.

5. The weight reduction program of claim 1 further comprising:
a predetermined amount of cyanocobalomine is given during said acute weight loss phase.

6. The weight reduction program of claim 2 further comprising:
a predetermined amount of cyanocobalomine is given during said acute weight loss phase.

7. The weight reduction program of claim 1 further comprising:
a predetermined amount of calcium pyruvate is taken during said acute weight loss phase.

8. The weight reduction program of claim 2 further comprising:
a predetermined amount of calcium pyruvate is taken during said acute weight loss phase.

9. The weight reduction program of claim 5 further comprising:
a predetermined amount of calcium pyruvate is taken during said acute weight loss phase.

10. The weight reduction program of claim 6 further comprising:
a predetermined amount of calcium pyruvate is taken during said acute weight loss phase.

11. The weight reduction program of claim 1 wherein:
said predetermined amount of at least one multiple vitamin-mineral supplement taken during said acute weight loss phase is decreased during said transitional phase.

12. The weight reduction program of claim 2 wherein:
said predetermined amount of at least one multiple vitamin-mineral supplement taken during said acute weight loss phase is decreased during said transitional phase.

13. The weight reduction program of claim 5 wherein:
said predetermined amount of at least one multiple vitamin-mineral supplement taken during said acute weight loss phase is decreased during said transitional phase.

14. The weight reduction program of claim 6 wherein:
said predetermined amount of at least one multiple vitamin-mineral supplement taken during said acute weight loss phase is decreased during said transitional phase.

15. The weight reduction program of claim 9 wherein:
said predetermined amount of at least one multiple vitamin-mineral supplement taken during said acute weight loss phase is decreased during said transitional phase.

16. The weight reduction program of claim 10 wherein:
said predetermined amount of at least one multiple vitamin-mineral supplement taken during said acute weight loss phase is decreased during said transitional phase.

17. The weight reduction program of claim 1 further comprising:
a predetermined amount of exercise is performed during said acute weight loss phase.

18. The weight reduction program of claim 1 further comprising:
a predetermined amount of exercise is performed during said long-term maintenance phase.

19. A weight reduction program for losing a predetermined amount of weight and maintaining a loss of said predetermined amount of weight comprising:
an acute weight loss phase wherein a user enters ketosis;
a predetermined amount of phenylalkylamine sympathomimetic amine is taken during said acute weight loss phase;
a predetermined amount of at least one multiple vitamin-mineral supplement is taken during said acute weight loss phase;
a predetermined amount of calcium pyruvate is taken during said acute weight loss phase;
a predetermined amount of cyanocobalomine is given during said acute weight loss phase;
a predetermined amount of exercise is performed during said acute weight loss phase;
a transitional phase wherein said predetermined loss of said predetermined amount of weight is maintained for a predetermined amount of time;
a predetermined amount of exercise is performed during said transitional phase;
said predetermined amount of at least one multiple vitamin-mineral supplement taken during said acute weight loss phase is decreased during said transitional phase; and
a long-term maintenance phase wherein a predetermined amount of calories equaling no more than ten times the user's goal weight per day is consumed and a predetermined amount of exercise is performed during said long-term maintenance phase.

20. A weight reduction method for losing a predetermined amount of weight and maintaining a loss of said predetermined amount of weight, said weight reduction program comprising an acute weight loss phase wherein a user enters ketosis; a predetermined amount of appetite suppressant is taken during said acute weight loss phase; a predetermined amount of at least one multiple vitamin-mineral supplement is taken during said acute weight loss phase; a transitional phase wherein said predetermined loss of said predetermined amount of weight is maintained for a predetermined amount of time; a predetermined amount of exercise is performed during said transitional phase; and a long-term maintenance phase wherein a predetermined amount of calories equaling no more than ten times a user's goal weight per day is consumed, said method comprising the steps of:
a. permitting a user's body to enter ketosis during said acute weight loss phase by limiting the user's diet;
b. ingesting said predetermined amount of appetite suppressant;
c. ingesting said predetermined amount of said at least one multiple vitamin-mineral supplement; and
d. performing a predetermined amount of exercise during said transitional phase.

21. The method of claim 20 wherein:
said predetermined amount of appetite suppressant is phenylalkylamine sympathomimetic amine.

22. The method of claim 20 further comprising a step after step a of:
injecting a predetermined amount of cyanocobalomine during said acute weight loss phase.

23. The method of claim 21 further comprising a step after step a of:
injecting a predetermined amount of cyanocobalomine during said acute weight loss phase.

24. The method of claim 20 further comprising a step after step a of:
ingesting a predetermined amount of calcium pyruvate during said acute weight loss phase.

25. The method of claim 21 further comprising a step after step a of:
ingesting a predetermined amount of calcium pyruvate during said acute weight loss phase.
